(19) United States
(12) Patent Application Publication
(10) Pub. No.: US 2023/0284909 A1
(43) Pub. Date: Sep. 14, 2023

(54) TEMPERATURE MANAGEMENT APPARATUS, SYSTEM AND METHOD, AND NON-TRANSITORY COMPUTER-READABLE MEDIUM STORING PROGRAM

(71) Applicant: NEC Corporation, Minato-ku, Tokyo (JP)

(72) Inventors: Yusuke Takahashi, Tokyo (JP); Ryuji Wakakusa, Tokyo (JP); Daiki Yamazaki, Tokyo (JP)

(73) Assignee: NEC Corporation, Minato-ku, Tokyo (JP)

(21) Appl. No.: 18/015,488

(22) PCT Filed: Jul. 17, 2020
(86) PCT No.: PCT/JP2020/027760

§ 371 Date: Jan. 10, 2023

(51) Int. Cl. A61B 5/01; A61B 5/00; A61B 5/1171

(52) U.S. Cl. CPC A61B 5/015; A61B 5/1171; A61B 5/7475; A61B 2560/0252; G16H 10/60

(57) ABSTRACT

A temperature management apparatus includes at least one memory storing instructions, and at least one processor. The at least one processor is configured to execute the instructions to obtain visible light image data, obtain thermal image data having an imaging range corresponding to at least a part of the visible light image, identify a person included in the visible light image data and the thermal image data, obtain attribute information from a storage apparatus, obtain temperature information, based on the thermal image data, set a threshold, based on the attribute information, determine whether the body surface temperature of the person exceeds the threshold or not and output determination result information.

### TECHNICAL FIELD

The present invention relates to a temperature management apparatus, system, method, and program.

### BACKGROUND ART

As a technology of managing the body surface temperatures of many people, a system using a visible light camera and an infrared camera has been known.

For example, an image processing system described in Patent Literature 1 identifies a person from a person image in a taken image, and measures heat from a facial image of the identified person. The image processing system then registers the facial image of the identified person, and the heat measured on the facial image of the person, in association with each other. The image processing system selects the facial image of the person with heat exceeding a predetermined threshold, and outputs the temporal change of the heat of the person identified by the selected facial image. The image processing system does not necessarily apply the threshold uniformly, but may sometimes change the threshold according to the identified person.

### CITATION LIST

### Patent Literature

Patent Literature 1 Japanese Unexamined Patent Application Publication No. 2012-235415

### SUMMARY OF INVENTION

### Technical Problem

However, the description about the aforementioned image processing system does not disclose in what cases and conditions for changing the threshold, or in what elements about which the threshold is changed. Accordingly, the technology described above has a possibility that an alert is issued to a person who requires no alert, and no alert is issued to a person who should be alerted.

The present disclosure has been made to solve such problems, and has an object to provide a temperature management apparatus, system, method and program that appropriately perform temperature management according to individual situations.

### Solution to Problem

A temperature management apparatus according to an example embodiment of the present disclosure includes visible light image data obtaining means, thermal image data obtaining means, identification means, attribute information control means, temperature information obtaining means, threshold setting means, and determination result output means. The visible light image data obtaining means obtains visible light image data that is data on a visible light image taken by a visible light camera. The thermal image data obtaining means obtains thermal image data that is data on an image taken by an infrared camera having an imaging range corresponding to at least a part of the visible light image. The identification means identifies a person included in the visible light image data and the thermal image data. The attribute information control means obtains attribute information on the identified person, from a storage apparatus configured to store the attribute information. The temperature information obtaining means obtains temperature information about a body surface temperature of the person, based on the thermal image data. The threshold setting means sets a threshold, based on the attribute information. The determination result output means determines whether the body surface temperature of the person exceeds the threshold or not and outputs determination result information based on a result of determination.

A temperature management method according to an example embodiment of the present disclosure causes a computer to execute the following method. The computer obtains visible light image data that is data on a visible light image taken by a visible light camera. The computer obtains thermal image data that is data on an image taken by an infrared camera having an imaging range corresponding to at least a part of the visible light image. The computer identifies a person included in the visible light image data and the thermal image data. The computer obtains attribute information on the identified person, from a storage apparatus configured to store the attribute information. The computer obtains temperature information about a body surface temperature of the person, based on the thermal image data. The computer sets a threshold, based on the attribute information. The computer determines whether the body surface temperature of the person exceeds the threshold or not and outputs determination result information based on a result of determination.

A program according to an example embodiment of the present disclosure causes a computer to execute the following steps. The computer obtains visible light image data that is data on a visible light image taken by a visible light camera. The computer obtains thermal image data that is data on an image taken by an infrared camera having an imaging range corresponding to at least a part of the visible light image. The computer identifies a person included in the visible light image data and the thermal image data. The computer obtains attribute information on the identified person, from a storage apparatus configured to store the attribute information. The computer obtains temperature information about a body surface temperature of the person, based on the thermal image data. The computer sets a threshold, based on the attribute information. The computer determines whether the body surface temperature of the person exceeds the threshold or not and outputs determination result information based on a result of determination.

### Advantageous Effects of Invention

This disclosure can provide a temperature management apparatus, system, method and program that are capable of appropriately performing temperature management according to individual situations.

### BRIEF DESCRIPTION OF THE DRAWINGS

FIG. 1 is a block diagram showing a configuration of a temperature management apparatus according to a first example embodiment.

FIG. 2 is a flowchart showing a temperature management method according to the first example embodiment.

FIG. 3 is a block diagram showing a configuration of a temperature management apparatus according to a second example embodiment.

FIG. 4 is a flowchart showing a temperature management method according to the second example embodiment.

FIG. 5 is a block diagram showing a configuration of a temperature management system according to a third example embodiment.

FIG. 6 is a block diagram showing a configuration of an authentication apparatus.

FIG. 7 is a flowchart showing a flow of a facial feature information registration process.

FIG. 8 is a flowchart showing a flow of a face authentication process.

FIG. 9 is a block diagram showing a configuration of a face authentication unit.

FIG. 10 is a block diagram showing a configuration of a user terminal.

FIG. 11 is a block diagram showing a configuration of a temperature management apparatus.

### DESCRIPTION OF EMBODIMENTS

Hereinafter, example embodiments of the present disclosure are described in detail with reference to the drawings. In each diagram, the same or corresponding elements are assigned the same reference signs. To clarify the description, redundant description is omitted as required.

### First Example Embodiment

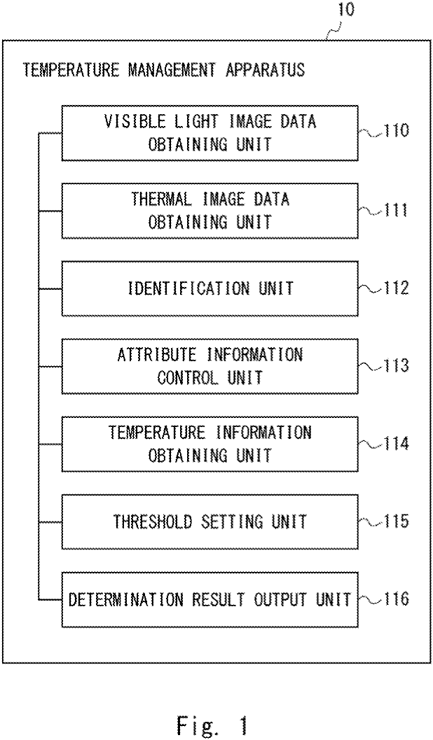

Referring to FIG. 1, the configuration of a temperature management apparatus is described. FIG. 1 is a block diagram showing the configuration of the temperature management apparatus 10 according to a first example embodiment. The temperature management apparatus 10 is connected to a communication network (hereinafter, the communication network is also simply called a network), and is connected to an imaging device, an authentication apparatus, a storage apparatus, and a terminal apparatus via the connected network.

The imaging device takes an image of scenery including a person, and supplies the temperature management apparatus 10 with visible light image data and thermal image data about the taken image of scenery. The imaging device includes a visible light camera, and an infrared camera. The visible light camera includes an imaging element that converts taken visible light into an electric signal. The visible light camera takes an image of a predetermined imaging range, and generates the visible light image data that is image data on the visible light image. The infrared camera includes an infrared imaging element that converts taken infrared light into an electric signal. The infrared camera takes an image of a predetermined imaging range, and generates the thermal image data that is image data on a thermal image.

The visible light camera and the infrared camera in the imaging device have imaging ranges that at least partially overlap with each other. In other words, the infrared camera has an imaging range corresponding to at least a part of a visible light image taken by the visible light camera. Preferably, the visible light camera and the infrared camera are fixed so as not to change their positional relationship. Such a configuration allows the imaging device to associate an object included in the visible light image data generated by the visible light camera with an object included in the thermal image data generated by the infrared camera.

The authentication apparatus receives the visible light image data from the temperature management apparatus 10, and performs authentication pertaining to the received visible light image data. The authentication apparatus performs authentication using the feature amount of a person extracted from the visible light image data, for example. More specifically, the authentication apparatus may perform authentication from a facial image of the person extracted from the visible light image data. The authentication apparatus may perform authentication on the basis of a display object worn by the person extracted from the visible light image data. The authentication apparatus supplies the temperature management apparatus 10 with a result of the authentication performed as described above.

The storage apparatus stores attribute information on a person, and provides the temperature management apparatus 10 with attribute information on the person pertaining to face authentication. The attribute information is stored in association with an identifier of the person, for example. The attribute information includes information that the temperature management apparatus 10 can use when setting a threshold. The attribute information may include, for example, profile information, such as on the age or gender of the person. The attribute information may include, for example, a threshold for the body surface temperature of the person. The attribute information may include basic data for calculating the threshold for the body surface temperature of the person. The basic data may be on the body surface temperature of the person (temperature history data) previously obtained, for example.

The attribute information may include behavior history information about a behavior history of the person. The behavior history information is data that associates date and time information with position information, and indicates when and where the person was present. For example, the behavior history information may include a history of position information and time information obtained using GNSS (Global Navigation Satellite System). The behavior history information may include information indicating presence adjacent to another apparatus based on information indicating that communication has been made with the other apparatus. The behavior history information may include information indicating check-in and check-out at a predetermined place. The behavior history information may include information indicating use of a predetermined transportation system. The attribute information includes the behavior history information, thereby allowing the temperature management apparatus 10 to set a threshold according to the behavior history of the person. For example, if a person has visited a specific area and the feature of an infection epidemic in the area relates to the body temperature, possible detection of a visit to the area from the behavior history of the person allows the temperature management apparatus 10 to set a threshold according thereto.

The terminal apparatus receives information about a determination result for a person pertaining to authentication, from the temperature management apparatus 10, and displays the received determination result. The information about the determination result received by the terminal apparatus indicates that the person pertaining to authentication has been actually authenticated, for example. The determination result received by the terminal apparatus may include, for example, what indicates that the body surface temperature of the person pertaining to authentication exceeds the threshold, and what indicates that the body surface temperature of the person pertaining to authentication does not exceed the threshold. The determination result may include an authentication ID of the user, the attribute information, and a result of determination of whether the body surface temperature of the person pertaining to the temperature information exceeds the threshold or not.

Note that the network may be any network irrespective of a wired or wireless one, and irrespective of the type of communication protocol. The temperature management apparatus 10 includes, as main components: a visible light image data obtaining unit 110; a thermal image data obtaining unit 111; an identification unit 112; an attribute information control unit 113; a temperature information obtaining unit 114; a threshold setting unit 115; and a determination result output unit 116.

The visible light image data obtaining unit 110 obtains data on a visible light image taken by the visible light camera included in the imaging device. The thermal image data obtaining unit 111 obtains data on a thermal image taken by the infrared camera included in the imaging device.

The identification unit 112 identifies the person imaged by the visible light camera and the infrared camera. Here, identification of the person means identification of the person included in images taken by the visible light camera and the infrared camera, and authentication of the identified person. The identification unit 112 extracts image data pertaining to an image of the person, from the visible light image data obtained by the visible light image data obtaining unit 110, and supplies the extracted image data to the authentication apparatus. The identification unit 112 then receives an authentication result from the authentication apparatus. If the authentication succeeds, the identification unit 112 can identify and authenticate the person imaged by the visible light camera and the infrared camera. That is, the identification unit 112 can identify a person included in the visible light image data and the thermal image data.

Note that in a case where the visible light camera images multiple people at the same time, the visible light image data includes an image of the multiple people. In this case, the identification unit 112 extracts and identifies each of the people from the visible light image data, and causes the authentication apparatus to authenticate each of the extracted people. Even if the visible light image data and the thermal image data include the multiple people as described above, the identification unit 112 identifies each predetermined person.

The attribute information control unit 113 obtains attribute information on the identified person, from a storage apparatus configured to store the attribute information. For example, the attribute information control unit 113 obtains an identifier of the identified person from the result of authentication received from the authentication apparatus, and reads the attribute information pertaining to the obtained identifier from the storage apparatus.

The temperature information obtaining unit 114 obtains temperature information about the body surface temperature of the identified person, from the thermal image data. The temperature information obtaining unit 114 identifies a person area in the thermal image data corresponding to the area where the person identified from the visible light image data has been imaged. The temperature information obtaining unit 114 extracts such a person area, and generates temperature information on the person identified on the basis of luminance values of the thermal image data in the extracted person area. The temperature information includes the body surface temperature of the person. The temperature information may be information about a part of the body of the person, and the body surface temperature, which are associated with each other. The temperature information may be image data on a thermal image that can be converted into the body surface temperature of the person.

Note that the temperature information obtaining unit 114 may further extract an area for obtaining temperature information in order to generate temperature information on the person, from the person area identified in the thermal image data. For example, the temperature information obtaining unit 114 may extract an area corresponding to a facial image of the person, and generate temperature information from thermal image data on the face of the person. The temperature information obtaining unit 114 may extract a forehead part of the person or an ear part of the person, instead of or together with the face of the person.

The temperature information obtaining unit 114 may use the highest temperature in the extracted thermal image data, as the temperature information. The temperature information obtaining unit 114 may calculate a predetermined statistic value from the extracted thermal image data, and use the calculated statistic value as the temperature information.

The threshold setting unit 115 sets the threshold from the attribute information on the person obtained by the attribute information control unit 113. The threshold is used to observe change in the physical condition of the person. For example, in consideration of the normal body temperature of the person, the threshold setting unit 115 sets the threshold so as to be capable of detecting deviation of the body temperature from the range of the normal body temperature of the person. In this case, the threshold setting unit 115 sets the threshold from the temperature history information on the person included in the attribute information. That is, the threshold setting unit 115 calculates the statistic value of the temperature in the temperature history information on the person, and sets the threshold on the basis of the calculated value. The statistic value is, for example, an average value, a variance, a standard deviation, etc. The average value may be a moving average, or a weighted average. Note that the number of thresholds may be any number only if the number is one or more. The threshold may be set on a side higher than the range of the normal body temperature, or on a side lower than the range of the normal body temperature. Multiple thresholds may be set on the side higher than the normal body temperature, for example. Such setting of the multiple thresholds allows the temperature management apparatus 10 to output various pieces of determination result information.

In a case where the attribute information control unit 113 obtains the profile information, that is, the attribute information includes the profile information, the threshold setting unit 115 can set the threshold using the profile information on the person. In general, the normal body temperatures of children are higher than the normal body temperatures of adults. Accordingly, the threshold setting unit 115 may set the threshold according to the age of the person.

In a case where the attribute information control unit 113 obtains the behavior history information, that is, the attribute information includes the behavior history information, the threshold setting unit 115 can set the threshold using the behavior history information on the person.

The determination result output unit 116 determines whether the body surface temperature of the person exceeds the threshold or not and outputs information about the determination result for the person (determination result information) based on a result of determination. For example, when the body surface temperature included in the temperature information on the person exceeds the threshold, the determination result output unit 116 outputs determination result information indicating that the body temperature of the person is possibly high. For example, when the body surface temperature included in the temperature information on the person further exceeds the threshold, the determination result output unit 116 outputs determination result information indicating an alert for the person. For example, when the body surface temperature included in the temperature information on the person does not exceed the threshold, the determination result output unit 116 outputs determination result information indicating absence of abnormality with respect to the person.

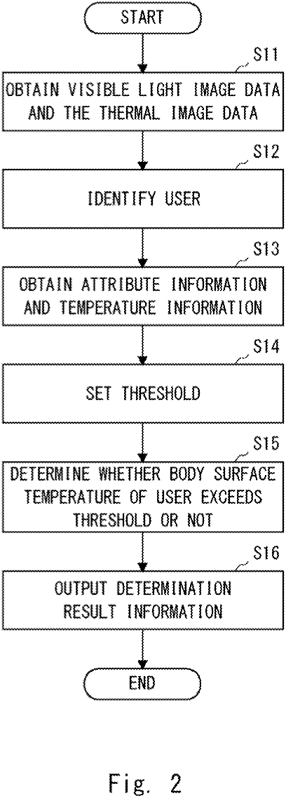

Next, referring to FIG. 2, the process of the temperature management apparatus 10 is described. FIG. 2 is a flowchart showing a temperature management method according to the first example embodiment.

First, the temperature management apparatus 10 obtains the visible light image data and the thermal image data from the imaging device via the network (step S11). In this case, the temperature management apparatus 10 may request the imaging device to transmit the image data, or accept the visible light image data and the thermal image data transmitted from the imaging device every predetermined period. Note that the timing of obtaining the visible light image data and the timing of obtaining the thermal image data may be the same as or different from each other. Note that it is preferable that the visible light image data and the thermal image data that are obtained each be in such a form that the corresponding relationship therebetween is recognizable.

Next, the identification unit 112 of the temperature management apparatus 10 identifies the person imaged by the visible light camera and the infrared camera (step S12). More specifically, for example, the identification unit 112 detects a facial image of the person included in the visible light image data, extracts the detected facial image, and causes the authentication apparatus to perform face authentication on the image data. At this time, for example, the temperature management apparatus 10 supplies the authentication apparatus with the facial image of the person, and requests the authentication apparatus to perform authentication.

The authentication apparatus stores pieces of identification information on multiple people, and respective pieces of facial feature information, in association with each other. For example, the authentication apparatus extracts a predetermined feature amount from the facial image of the person included in the image data, and associates the extracted feature amount with the identification information on the person. If the authentication succeeds, the authentication apparatus supplies the temperature management apparatus 10 with the identification information on the person associated with the received image data. Accordingly, the identification unit 112 identifies the person on the basis of the detected facial image and the result of authentication pertaining to the facial image.

Next, the temperature management apparatus 10 obtains the attribute information and the temperature information (step S13). More specifically, the attribute information control unit 113 of the temperature management apparatus 10 reads the attribute information on the identified person from the storage apparatus. The temperature information obtaining unit 114 of the temperature management apparatus 10 obtains the temperature information pertaining to the identified person.

Next, the threshold setting unit 115 of the temperature management apparatus 10 sets the threshold for the identified person on the basis of the obtained attribute information (step S14). After setting the threshold, the threshold setting unit 115 supplies the set threshold to the determination result output unit 116.

Next, the determination result output unit 116 of the temperature management apparatus 10 determines whether the body surface temperature of the person exceeds the threshold or not on the basis of the set threshold and the obtained temperature information (step S15).

Next, the determination result output unit 116 generates determination result information according to the result of the determination, and outputs the generated determination result information (step S16). After the determination result information is output, the temperature management apparatus 10 finishes the processing. Note that if the temperature management apparatus 10 continuously obtains the visible light image data and the thermal image data from the imaging device, the processes from step S11 are repeated.

The temperature management apparatus 10 according to the first example embodiment has thus been described. Note that even if the imaged person is not successfully authenticated, the temperature management apparatus 10 may detect the body surface temperature of the person included in the image, and execute the process of outputting the determination result information using the predetermined threshold.

Note that the temperature management apparatus 10 includes a processor and a storage apparatus, which are components not shown. The storage apparatus included in the temperature management apparatus 10 encompasses a storage apparatus that includes non-volatile memories, such as a flash memory and an SSD (Solid State Drive). In this case, the storage apparatus included in the temperature management apparatus 10 may store the attribute information; the attribute information on the person pertaining to the successful face authentication may be read from such a storage apparatus.

The storage apparatus included in the temperature management apparatus 10 stores a computer program (hereinafter, also simply called a program) for executing the temperature management method according to this example embodiment. The processor causes the memory to read the computer program from the storage apparatus, and executes the program.

Each component included in the temperature management apparatus 10 may be achieved in dedicated hardware. A part, some or all of the components may be achieved by general or dedicated circuitry, a processor or the like, or a combination thereof. The component(s) may be made up of a single chip, or multiple chips connected to each other via a bus. Each configuration element of each apparatus may be partially or entirely achieved by the combination of the circuitry or the like and the program described above. A CPU (central processing unit), a GPU (graphics processing unit), an FPGA (field-programmable gate array) or the like may be adopted as the processor.

In a case where each configuration element of the temperature management apparatus 10 is partially or entirely made up of multiple information processing apparatuses, circuitry or the like, the multiple information processing apparatuses, circuitry or the like may be arranged in a centralized manner or arranged in a distributed manner. For example, the information processing apparatuses, circuitry or the like may be achieved in a mode where a client server system, a cloud computer system and the like are connected via a communication network. The function of the temperature management apparatus 10 may be provided in a SaaS (Software as a Service) form.

The temperature management apparatus 10 described above identifies the person to thereby obtain the attribute information on the identified person, sets the threshold on a person-by-person basis, and performs temperature management on a person-by-person basis. Consequently, the temperature management apparatus 10 can perform the temperature management according to the individual situations of each person. That is, this example embodiment can provide a temperature management apparatus, system, method and program that are capable of appropriately performing temperature management according to individual situations.

### Second Example Embodiment

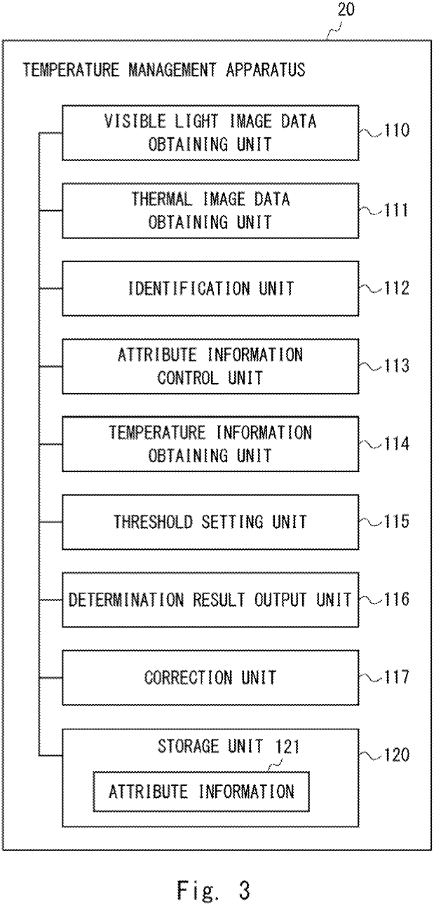

Next, a second example embodiment is described. A temperature management apparatus according to the second example embodiment is different from that of the first example embodiment in that a correction unit and a storage unit are included. FIG. 3 is a block diagram showing the configuration of the temperature management apparatus according to the second example embodiment. The temperature management apparatus 20 shown in FIG. 3 includes, as main components: a visible light image data obtaining unit 110; a thermal image data obtaining unit 111; an identification unit 112; an attribute information control unit 113; a temperature information obtaining unit 114; a threshold setting unit 115; a determination result output unit 116; a correction unit 117; and a storage unit 120.

The correction unit 117 detects the ambient temperature around the person pertaining to the temperature information, and corrects the temperature information according to the detected ambient temperature. More specifically, the correction unit 117 detects the ambient temperature around the person from the thermal image data in an area where the person and therearound have been imaged. When the ambient temperature is relatively higher than a predetermined reference temperature, the correction unit 117 corrects the body surface temperature of the person to a low value. On the contrary, when the ambient temperature is relatively lower than the predetermined reference temperature, the correction unit 117 corrects for increasing the body surface temperature. The correction unit 117 preliminarily has reference data on the ambient temperature in order to perform such correction. The correction unit 117 may use history information on the ambient temperature, as such reference data. Note that the correction unit 117 may obtain temperature data from a temperature sensor provided adjacent to the person or the infrared camera, and correct the temperature information according to the obtained temperature data. Note that upon detection that the person wears a mask, the correction unit 117 may correct the body surface temperature so as to decrease. This is because in a case where the person wears a mask, the body surface temperature tends to be relatively higher than that in a case where they wear no mask. The correction unit 117 can detect that the person wears a mask from facial images included in the visible light image data and the thermal image data.

The storage unit 120 is a storage apparatus that includes a non-volatile memory, and stores at least attribute information 121. The attribute information 121 is associated with the identification information on the person, and may include index information accompanying the corresponding attribute information. The index information is accompanying information for allowing multiple pieces of attribute information to be extracted and retrieved on a layer-by-layer basis.

The attribute information control unit 113 according to this example embodiment updates the attribute information 121 using the temperature information obtained by the temperature information obtaining unit 114. More specifically, the attribute information control unit 113 adds the temperature information to the attribute information 121 on the basis of the temperature information obtained by the temperature information obtaining unit 114 and the attribute information 121 obtained by the attribute information control unit 113, thereby generating new attribute information 121. That is, the attribute information control unit 113 updates the attribute information 121. The attribute information control unit 113 then writes the updated attribute information 121 in the storage unit 120. Accordingly, the temperature management apparatus 20 performs temperature management using the attribute information that always includes new temperature information in the attribute information on the person.

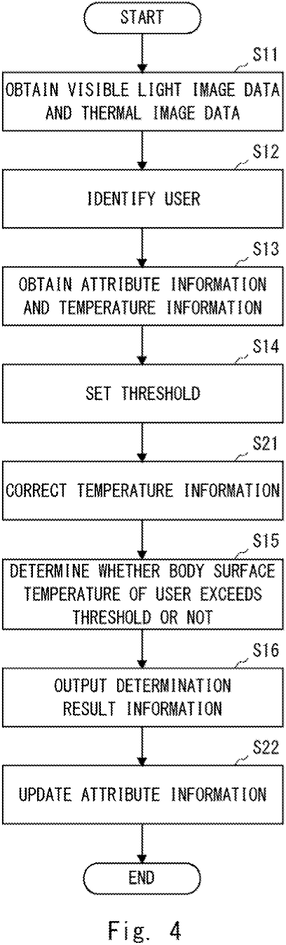

Next, the process of the temperature management apparatus 20 is described. FIG. 4 is a flowchart showing an example of a temperature management method according to the second example embodiment. The flowchart of FIG. 4 is different from the flowchart in the first example embodiment in that step S21 is included at step S14 and in that step S22 is included after step S16. Note that in the following flowchart, description of processes equivalent to the processes in the aforementioned flowchart is appropriately omitted.

The threshold setting unit 115 of the temperature management apparatus 20 sets the threshold for the identified person on the basis of the attribute information obtained in step S13 (step S14). After setting the threshold, the threshold setting unit 115 supplies the set threshold to the determination result output unit 116.

Next, the correction unit 117 of the temperature management apparatus 20 corrects the temperature information obtained by the temperature information obtaining unit 114 (step S21). The correction unit 117 supplies the corrected temperature information to the determination result output unit 116.

Next, the determination result output unit 116 of the temperature management apparatus 10 determines whether the body surface temperature of the person exceeds the threshold or not on the basis of the set threshold and the obtained corrected temperature information (step S15). Furthermore, the temperature management apparatus 10 generates determination result according to the result of the determination, and outputs the generated determination result information (step S16).

Next, the attribute information control unit 113 updates the attribute information using the corrected temperature information, and writes the updated attribute information in the storage unit 120 (step S22). After step S22 is finished, the temperature management apparatus 10 finishes the processing. Note that if the temperature management apparatus 10 continuously obtains the visible light image data and the thermal image data from the imaging device, the processes from step S11 are repeated.

The flowchart of FIG. 4 has thus been described. Note that in the temperature management method of FIG. 4, steps S14 and S21 may be replaced with each other, and steps S14 and S21 may be executed in parallel. In the temperature management method of FIG. 4, steps S16 and S22 may be replaced with each other, and steps S16 and S22 may be executed in parallel.

The second example embodiment has thus been described. The temperature management apparatus 20 described above identifies the person, and obtains the attribute information on the identified person, thereby performing temperature management on a person-by-person basis. The temperature management apparatus 20 can perform temperature management following change in temperature information, by updating the attribute information on the basis of the detected body surface temperature of the person. Consequently, the second example embodiment can provide a temperature management apparatus, system, method and program that are capable of appropriately performing temperature management according to individual situations.

### Third Example Embodiment

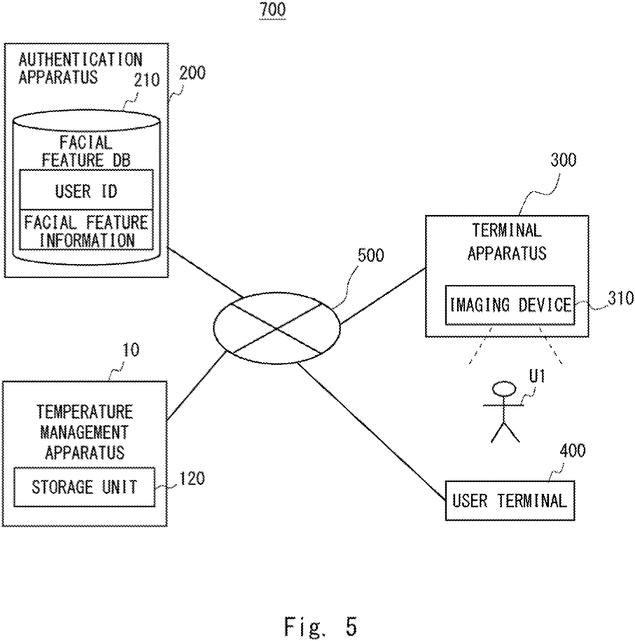

Next, a third example embodiment is described. The third example embodiment is an example of a system including the temperature management apparatus 10 according to the second example embodiment described above. FIG. 5 is a block diagram showing the configuration of a temperature management system 700 according to the third example embodiment. The temperature management system 700 includes the temperature management apparatus 10, an authentication apparatus 200, a terminal apparatus 300, and a user terminal 400. The temperature management apparatus 10, the authentication apparatus 200, the terminal apparatus 300, and the user terminal 400 are connected to each other via a network 500. Note that in this example embodiment, the temperature management apparatus 10 includes the storage unit 120. Description redundant also in that of the first example embodiment is appropriately omitted.

The temperature management apparatus 10 identifies a person U1 on the basis of a visible light image where the person U1 has been taken, and outputs a predetermined message according to whether the temperature information on the person U1 exceeds the threshold or not. The temperature management apparatus 10 is, for example, a server apparatus implemented by a computer.

The authentication apparatus 200 is an apparatus configured to perform face authentication. The authentication apparatus 200 includes a facial feature DB 210. The facial feature DB 210 is a facial feature database that stores user IDs on individual people and pieces of facial feature information on the respective people in association with each other. Note that the facial feature DB 210 is an example of a facial feature information storage unit.

The terminal apparatus 300 includes an imaging device 310. The imaging device 310 is the imaging device described in the first example embodiment, and includes the visible light camera and the infrared camera. The terminal apparatus 300 is installed at a place where people using a facility, such as a gateway or a gate of a facility on which temperature management is performed, passes. The terminal apparatus 300 images the face of the person U1 visiting the facility, and transmits a face authentication request including the taken image, to the temperature management apparatus 10. If such face authentication succeeds, the terminal apparatus 300 receives information about the determination result for the person U1, and displays a predetermined determination result. Note that a plurality of terminal apparatuses 300 may be installed in such a facility. The imaging device 310 included in the terminal apparatus 300 may be integrated in the terminal apparatus 300 or be separately configured.

The user terminal 400 is an information processing apparatus that the person U1 operates. The user terminal 400 is a mobile terminal having a communication function, such as a smartphone, a tablet, or a note PC, for example. The user terminal 400 receives a predetermined determination result from the temperature management apparatus 10. The user terminal 400 transmits a behavior history or the like of the user to the temperature management apparatus 10.

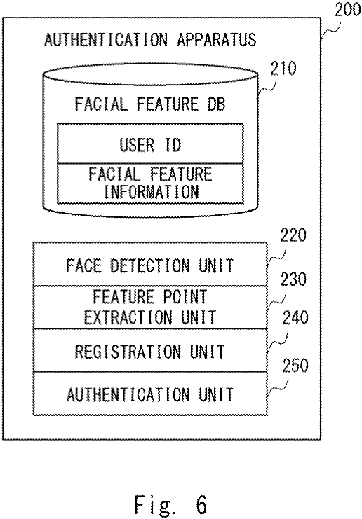

Next, referring to FIG. 6, the configuration of the authentication apparatus 200 is described in detail. FIG. 6 is a block diagram showing the configuration of the authentication apparatus 200. The authentication apparatus 200 includes the facial feature DB 210, a face detection unit 220, a feature point extraction unit 230, a registration unit 240, and an authentication unit 250.

The facial feature DB 210 is a facial feature database that stores user IDs on individual people and pieces of facial feature information on the respective people in association with each other. The face detection unit 220 detects a face area included in a taken image, and outputs the area to the feature point extraction unit 230. The feature point extraction unit 230 extracts feature points from the face area detected by the face detection unit 220, and outputs the facial feature information to the registration unit 240. The facial feature information is a set of the extracted feature points.

When the facial feature information is registered, the registration unit 240 newly issues a user ID. The registration unit 240 registers the issued user ID, and the facial feature information extracted from the registered image, in association with each other, in the facial feature DB 210. The authentication unit 250 matches the facial feature information extracted from the facial image against the facial feature information in the facial feature DB 210. The authentication unit 250 determines that the face authentication succeeds if the facial feature information coincides, and determines that the face authentication fails if the facial feature information does not coincide. The authentication unit 250 returns success or failure to the temperature management apparatus 10. Coincidence or non-coincidence of the facial feature information corresponds to success or failure of authentication. If the face authentication succeeds, the authentication unit 250 identifies the user ID associated with the successful facial feature information, and returns the identified user ID, and an authentication result including the fact of success of authentication, to the temperature management apparatus 10.

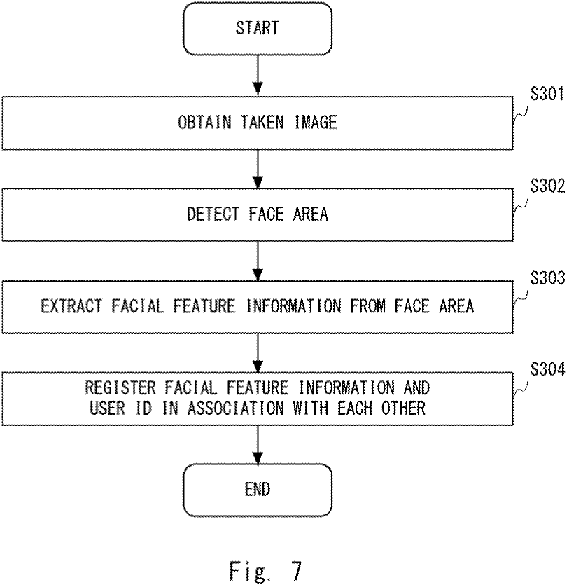

Referring to FIG. 7, the operation of the authentication apparatus 200 during registration of the user ID and the facial feature information in the facial feature DB 210 is described. FIG. 7 is a flowchart showing the flow of the facial feature information registration process.

When the facial feature information is registered, a facial information registration terminal (not shown) takes an image including the face of a person as an image for registration, and issues a facial feature information registration request to the authentication apparatus 200 via the network 500. The facial information registration terminal may be, for example, an information processing apparatus, such as a personal computer, a smartphone, or a tablet terminal. The facial information registration terminal may be the user terminal 400. The facial feature information registration request includes the image for registration. First, the authentication apparatus 200 obtains the image for registration included in the facial feature information registration request, through the network 500 from the facial information registration terminal (step S301).

Next, the face detection unit 220 detects the face area included in the image for registration (step S302), and outputs the detected face area to the feature point extraction unit 230. Next, the feature point extraction unit 230 extracts feature points from the face area, and outputs the facial feature information to the registration unit 240 (step S303). Next, the registration unit 240 issues the user ID corresponding to the output facial feature information, and registers the user ID and the facial feature information in association with each other in the facial feature DB 210 (step S304).

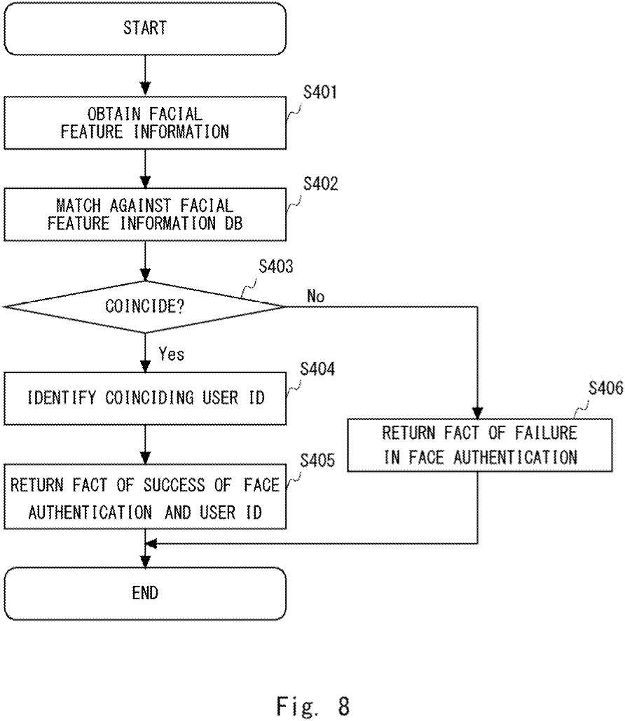

Referring to FIG. 8, the operation of the authentication apparatus 200 during face authentication is described. FIG. 8 is a flowchart showing the flow of a face authentication process. When the face authentication is performed, the terminal apparatus 300 takes an image including the face of a person as an image for authentication, and issues a face authentication request to the temperature management apparatus 10 via the network 500. The face authentication request includes the image for authentication. First, the temperature management apparatus 10 transmits the face area or the facial feature information extracted from the image for authentication, to the authentication apparatus 200. The feature point extraction unit 230 extracts the facial feature information from the received face area, or receives the facial feature information, thereby obtaining the facial feature information (step S401).

Next, the authentication unit 250 matches the obtained facial feature information against the facial feature DB 210 (step S402). If the facial feature information coincides (step S403: YES), the authentication unit 250 identifies the user ID of the person on which the facial feature information coincides (step S404), and returns the fact of success of face authentication and the identified user ID to the temperature management apparatus 10 (step S405). If there is no coinciding facial feature information (step S403: NO), the authentication unit 250 returns the fact of failure in face authentication to the temperature management apparatus 10 (step S406). The authentication apparatus 200 can estimate the age segment and the gender on the basis of the facial feature information. In step S406, the authentication unit 250 includes the estimated age segment and gender in a reply, and returns the reply to the temperature management apparatus 10.

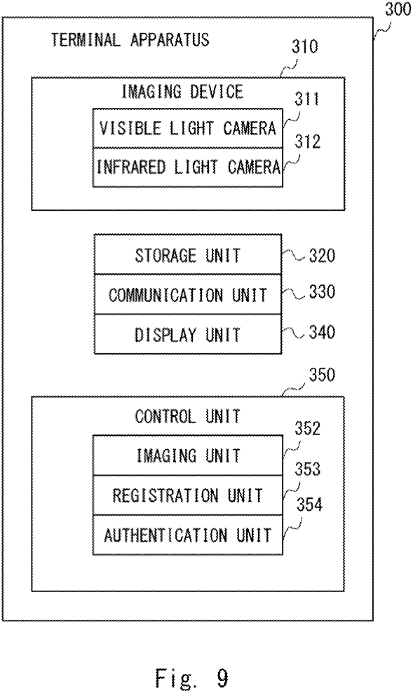

Next, referring to FIG. 9, the configuration of the terminal apparatus 300 is described in detail. The terminal apparatus 300 includes, as main hardware components: the imaging device; a display device; and a computer. FIG. 9 is a block diagram showing the configuration of the terminal apparatus 300. The terminal apparatus 300 includes, as main components: the imaging device 310; a storage unit 320; a communication unit 330; a display unit 340; and a control unit 350. The imaging device 310 includes a visible light camera 311, and an infrared camera 312.

The storage unit 320 is a storage apparatus configured to store a program for achieving each function of the terminal apparatus 300. The communication unit 330 is a communication interface with the network 500. The display unit 340 is a display device configured to display a message supplied from the temperature management apparatus 10. The control unit 350 controls hardware included in the terminal apparatus 300. The control unit 350 includes an imaging unit 352, a registration unit 353, and an authentication unit 354.

The imaging unit 352 issues an imaging request to the imaging device 310. The imaging device 310 takes a visible light image and a thermal image that include the face of a person. The visible light image taken by the imaging device 310 is used as an image for registration and an image for authentication. The registration unit 353 transmits the facial feature information registration request to the authentication apparatus 200 via the network 500. The facial feature information registration request includes the image for registration taken by the imaging device 310. The authentication unit 354 transmits a face authentication request to the temperature management apparatus 10 via the network 500. The face authentication request includes the image for authentication taken by the imaging device 310. The authentication unit 354 receives information about an authentication result for the person pertaining to face authentication from the temperature management apparatus 10, and displays the received authentication result on the display unit 340.

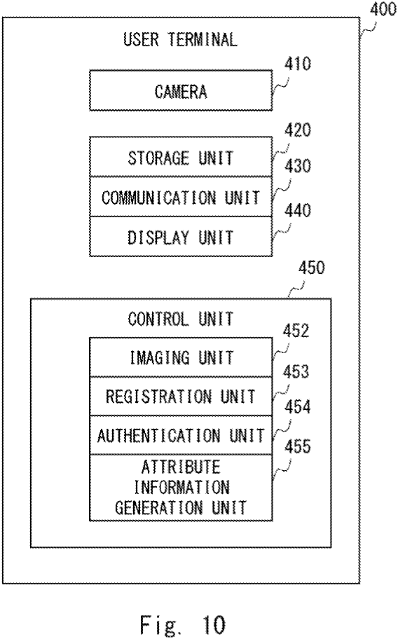

Next, referring to FIG. 10, the configuration of the user terminal 400 is described in detail. FIG. 10 is a block diagram showing the configuration of the user terminal 400. The user terminal 400 includes, as main components: a camera 410; a storage unit 420; a communication unit 430; a display unit 440; and a control unit 450. The control unit 450 includes an imaging unit 452, a registration unit 453, an authentication unit 454, and an attribute information generation unit. The camera 410 takes a visible light image.

The attribute information generation unit 455 included in the control unit 450 generates attribute information on a person (terminal user) having the user terminal. The attribute information generated by the user terminal 400 includes a user ID for allowing the temperature management apparatus 10 to identify the user terminal, and personal attribute information associated with the user ID. The personal attribute information can include profile information, such as the name and gender of the terminal user.

The personal information may include about the body temperature of the terminal user. The information about the body temperature of the terminal user may be input by the terminal user themself, or be body temperature data on the terminal user obtained from a thermometer, not shown.

The personal attribute information may include behavior history information. The behavior history information includes a time, and position information at the time detected by the user terminal 400 every predetermined period, respectively through a clock function (not shown) and a position information detection function (not shown) that the user terminal 400 has, for example.

The user terminal 400 is communicably connected to the temperature management apparatus 10, and can supply the temperature management apparatus 10 with attribute information generated by the attribute information generation unit 455. Note that in this case, the temperature management apparatus 10 may integrate the received attribute information with the attribute information having already been stored in the storage unit 120. The illustrated other functions of the user terminal 400 are identical or similar to the respective functions that the terminal apparatus 300 has. Accordingly, description thereof is herein omitted.

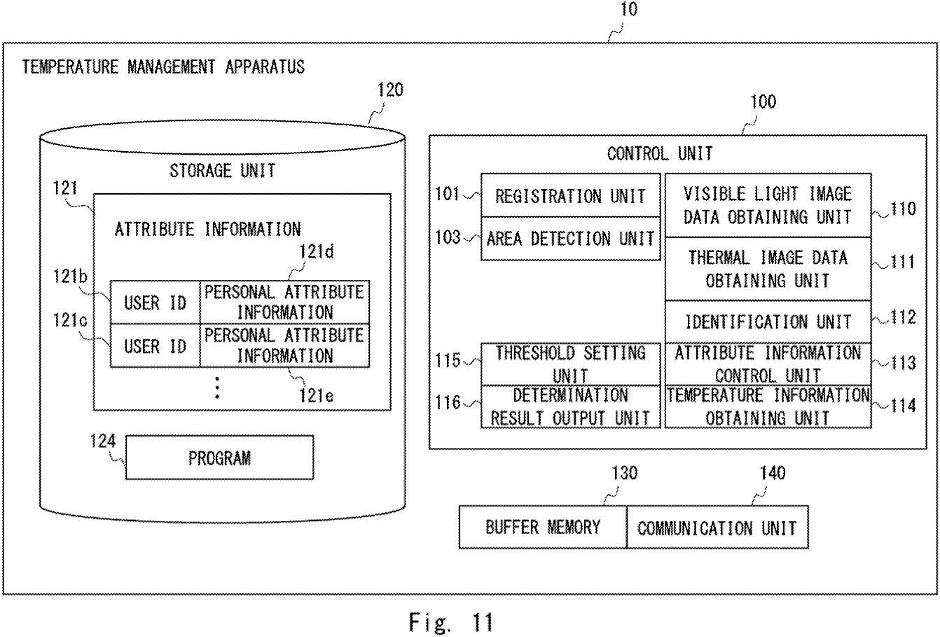

Next, referring to FIG. 11, the configuration of the temperature management apparatus 10 is described in detail. FIG. 11 is a block diagram showing the configuration of the temperature management apparatus 10. The temperature management apparatus 10 includes a control unit 100, a storage unit 120, a buffer memory 130, and a communication unit 140.

The storage unit 120 is a non-volatile storage apparatus configured to store at least the attribute information 121 and a program 124. The attribute information 121 is a set of the user IDs of the people and pieces of attribute information associated with the respective people. The user IDs are identifiers allowing the respective people to be identified. Pieces of personal attribute information 121d and 121e are associated with the respective user IDs 121b and 121c. That is, in the storage unit 120, the pieces of identification information on the people are associated with pieces of personal attribute information on the respective people. The pieces of personal attribute information 121d and 121e are, for example, the name, gender, age, behavior history and the like. However, there is no limitation thereto. The program 124 is a computer program where an information control process according to this example embodiment is implemented.

The buffer memory 130 is a memory area that temporarily stores the processing content of the control unit 100, and is, for example, a volatile storage apparatus, such as a RAM (Random Access Memory). The communication unit 140 is an interface that communicates with the outside of the temperature management apparatus 10.

The control unit 100 includes a registration unit 101, an area detection unit 103, a visible light image data obtaining unit 110, a thermal image data obtaining unit 111, an identification unit 112, an attribute information control unit 113, a temperature information obtaining unit 114, a threshold setting unit 115, and a determination result output unit 116. The control unit 100 is a control device configured to control the operation of the temperature management apparatus 10, and is, for example, a processor, such as a CPU. The control unit 100 reads the program 124 from the storage unit 120 into the buffer memory 130, and executes this program. Accordingly, the control unit 100 achieves each function in the configuration that the control unit 100 has.

The registration unit 101 receives the registration request issued by the terminal apparatus 300 or the user terminal 400, via the network 500. The registration unit 101 issues a user ID corresponding to the user ID included in the received registration request. The registration unit 101 associates the user ID with the personal attribute information corresponding to the individual user ID and generates the attribute information 121, and registers the generated attribute information 121 in the storage unit 120.

The area detection unit 103 detects the face area from the visible light image data. For example, the area detection unit 103 analyzes the visible light image data, and recognizes the face area of the user. When the face area is identified in the visible light image data, the identification unit 112 causes the authentication apparatus 200 to perform face authentication for each identified face area, using the facial feature information.

The attribute information control unit 113 receives update information from the user terminal, and reflects the update information in the attribute information.

The third example embodiment has thus been described. The temperature management system 700 according to the third example embodiment is not limited to that having the configuration described above. The determination result output unit 116 of the temperature management apparatus 10 is communicably connected to the user terminal 400, and outputs the determination result to the user terminal 400. Accordingly, even when multiple people are imaged at the same time, the temperature management system 700 can securely notify these individual people in the determination result. Even in situations where multiple people are imaged seamlessly and continuously, the temperature management system 700 can securely notify the individual people of the determination result.

The temperature management system 700 may have a configuration including the temperature management apparatus 20 described above instead of the temperature management apparatus 10.

The third example embodiment has thus been described. The third example embodiment can provide a temperature management apparatus, system, method and program that are capable of appropriately performing temperature management according to individual situations.

Note that the program described above can be stored using any of various non-transitory computer-readable media, and be supplied to the computer. The non-transitory computer-readable media encompass various types of tangible recording media. Examples of the non-transitory computer-readable media include magnetic recording media (e.g., a flexible disk, a magnetic tape, and a hard disk drive), magneto-optical recording media (e.g., a magneto-optical disk), a CD-ROM (read only memory), a CD-R, a CD-R/W, a semiconductor memory (e.g., a mask ROM, a PROM (programmable ROM), an EPROM (erasable PROM), a flash ROM, and a RAM (random access memory)). The program may be supplied to the computer through any of various types of transitory computer-readable media. Examples of the transitory computer-readable media include an electric signal, an optical signal, and electromagnetic waves. The transitory computer-readable media can supply the program to the computer via a wired communication path, such as electric wires and optical fibers, or a wireless communication path.

Note that the present invention is not limited to the example embodiments described above, and can be appropriately changed in a range without departing from the spirit.

A part, some or all of the aforementioned example embodiments can be described as in the following supplementary notes. However, there is no limitation to the following.

### (Supplementary Note 1)

A temperature management apparatus, comprising:
visible light image data obtaining means for obtaining visible light image data that is data on a visible light image taken by a visible light camera;
thermal image data obtaining means for obtaining thermal image data that is data on an image taken by an infrared camera having an imaging range corresponding to at least a part of the visible light image;
identification means for identifying a person included in the visible light image data and the thermal image data;
attribute information control means for obtaining attribute information on the identified person, from a storage apparatus configured to store the attribute information;
temperature information obtaining means for obtaining temperature information about a body surface temperature of the person, based on the thermal image data;
threshold setting means for setting a threshold, based on the attribute information; and
determination result output means for determining whether the body surface temperature of the person exceeds the threshold or not and outputting determination result information based on a result of determination.

### (Supplementary Note 2)

The temperature management apparatus according to Supplementary note 1, wherein

the threshold setting means sets the threshold, based on temperature history information that is a history of the body surface temperature of the person and is included in the attribute information.

### (Supplementary Note 3)

The temperature management apparatus according to Supplementary note 2, wherein

the threshold setting means sets the threshold, based on a statistic value of the temperature in the temperature history information.

### (Supplementary Note 4)

The temperature management apparatus according to any one of Supplementary notes 1 to 3, wherein
the attribute information control means obtains profile information on the person, as the attribute information, and
the threshold setting means sets the threshold, based on the profile information.

### (Supplementary Note 5)

The temperature management apparatus according to Supplementary note 4, wherein

the attribute information control means obtains, as the attribute information, the profile information including at least one of a gender or an age of the person.

### (Supplementary Note 6)

The temperature management apparatus according to any one of Supplementary notes 1 to 5, wherein
the attribute information control means obtains the attribute information including a behavior history of the person, and
the threshold setting means sets the threshold, based on the behavior history.

### (Supplementary Note 7)

The temperature management apparatus according to any one of Supplementary notes 1 to 6, further comprising

correction means for detecting an ambient temperature around the person pertaining to the temperature information, and performing correction of the temperature information, based on the detected ambient temperature.

### (Supplementary Note 8)

The temperature management apparatus according to Supplementary note 7, wherein

the correction means performs the correction, based on the thermal image data on an area where an image around the person has been taken.

### (Supplementary Note 9)

The temperature management apparatus according to Supplementary note 7, wherein

the correction means obtains temperature data from a temperature sensor provided adjacent to the person or the infrared camera, and performs the correction.

### (Supplementary Note 10)

The temperature management apparatus according to any one of Supplementary notes 1 to 9, wherein

the attribute information control means updates the attribute information, based on the temperature information.

### (Supplementary Note 11)

The temperature management apparatus according to any one of Supplementary notes 1 to 10, wherein

the determination result output means is communicably connected to a person terminal used by the person, and outputs the determination result information to the person terminal.

### (Supplementary Note 12)

The temperature management apparatus according to any one of Supplementary notes 1 to 11, wherein

the attribute information control means receives update information from a person terminal used by the person, and reflects the update information in the attribute information.

### (Supplementary Note 13)

A temperature management system, comprising:
the temperature management apparatus according to any one of Supplementary notes 1 to 12; and
a storage apparatus configured to associate the person and the attribute information with each other, and store the associated information.

### (Supplementary Note 14)

A temperature management system, comprising:
the temperature management apparatus according to any one of Supplementary notes 1 to 12; and
an authentication apparatus configured to receive the visible light image data from the temperature management apparatus, and authenticate the person.

### (Supplementary Note 15)

A temperature management method executed by a computer, the method comprising:
obtaining visible light image data that is data on a visible light image taken by a visible light camera;
obtaining thermal image data that is data on an image taken by an infrared camera having an imaging range corresponding to at least a part of the visible light image;
identifying a person included in the visible light image data and the thermal image data;
obtaining attribute information on the identified person, from a storage apparatus configured to store the attribute information;
obtaining temperature information about a body surface temperature of the person, based on the thermal image data;
setting a threshold, based on the attribute information; and
determining whether the body surface temperature of the person exceeds the threshold or not and outputting determination result information based on a result of determination.

### (Supplementary Note 16)

A non-transitory computer-readable medium storing an information control program causing a computer to execute:
a process of obtaining visible light image data that is data on a visible light image taken by a visible light camera;
a process of obtaining thermal image data that is data on an image taken by an infrared camera having an imaging range corresponding to at least a part of the visible light image;
a process of identifying a person included in the visible light image data and the thermal image data;
a process of obtaining attribute information on the identified person, from a storage apparatus configured to store the attribute information;
a process of obtaining temperature information about a body surface temperature of the person, based on the thermal image data;
a process of setting a threshold, based on the attribute information; and
a process of determining whether the body surface temperature of the person exceeds the threshold or not and outputting determination result information based on a result of determination.

### REFERENCE SIGNS LIST

10 Temperature management apparatus
20 Temperature management apparatus
100 Control unit
101 Registration unit
103 Area detection unit
110 Visible light image data obtaining unit
111 Thermal image data obtaining unit
112 Identification unit
113 Attribute information control unit
114 Temperature information obtaining unit
115 Threshold setting unit
116 Determination result output unit
117 Correction unit
120 Storage unit
121 Attribute information
130 Buffer memory
140 Communication unit
200 Authentication apparatus
210 Facial feature DB
220 Face detection unit
230 Feature point extraction unit
240 Registration unit
250 Authentication unit
300 Terminal apparatus
310 Imaging device
311 Visible light camera
312 Infrared camera
320 Storage unit
330 Communication unit
340 Display unit
350 Control unit
400 User terminal
420 Storage unit
430 Communication unit
440 Display unit
450 Control unit
455 Attribute information generation unit
500 Network
700 Temperature management system

What is claimed is:

1. A temperature management apparatus, comprising:
at least one memory storing instructions, and
at least one processor configured to execute the instructions to;
obtain visible light image data that is data on a visible light image taken by a visible light camera;
obtain thermal image data that is data on an image taken by an infrared camera having an imaging range corresponding to at least a part of the visible light image;
identify a person included in the visible light image data and the thermal image data;
obtain attribute information on the identified person, from a storage apparatus configured to store the attribute information;
obtain temperature information about a body surface temperature of the person, based on the thermal image data;
set a threshold, based on the attribute information; and
determine whether the body surface temperature of the person exceeds the threshold or not and output determination result information about a result of determination.

2. The temperature management apparatus according to claim 1, wherein
the at least one processor is further configured to execute the instruction to
set the threshold, based on temperature history information that is a history of the body surface temperature of the person and is included in the attribute information.

3. The temperature management apparatus according to claim 2, wherein
the at least one processor is further configured to execute the instruction to
set the threshold, based on a statistic value of the temperature in the temperature history information.

4. The temperature management apparatus according to claim 1, wherein
the at least one processor is further configured to execute the instructions to
obtain profile information on the person, as the attribute information, and
set the threshold, based on the profile information.

5. The temperature management apparatus according to claim 4, wherein
the at least one processor is further configured to execute the instruction to
obtain, as the attribute information, the profile information including at least one of a gender or an age of the person.

6. The temperature management apparatus according to claim 1, wherein
the at least one processor is further configured to execute the instructions to
obtain the attribute information including a behavior history of the person, and
set the threshold, based on the behavior history.

7. The temperature management apparatus according to claim 1,
the at least one processor is further configured to execute the instructions to
detect an ambient temperature around the person pertaining to the temperature information, and perform correction of the temperature information, based on the detected ambient temperature.

8. The temperature management apparatus according to claim 7, wherein
the at least one processor is further configured to execute the instruction to
perform the correction, based on the thermal image data on an area where an image around the person has been taken.

9. The temperature management apparatus according to claim 7, wherein
the at least one processor is further configured to execute the instructions to
obtain temperature data from a temperature sensor provided adjacent to the person or the infrared camera, and perform the correction.

10. The temperature management apparatus according to claim 1, wherein
the at least one processor is further configured to execute the instruction to
update the attribute information, based on the temperature information.

11. The temperature management apparatus according to claim 1, wherein
the at least one processor is further configured to execute the instructions to
communicably connect to a user terminal used by the person, and output the determination result information to the user terminal.

12. The temperature management apparatus according to claim 1, wherein
the at least one processor is further configured to execute the instructions to
receive update information from a user terminal used by the person, and reflect the update information in the attribute information.

13. A temperature management system, comprising:
the temperature management apparatus according to claim 1; and
a storage apparatus configured to associate the person and the attribute information with each other, and store the associated information.

14. A temperature management system, comprising:
the temperature management apparatus according to claim 1; and
an authentication apparatus configured to receive the visible light image data from the temperature management apparatus, and authenticate the person.

15. A temperature management method executed by a computer, the method comprising:
obtaining visible light image data that is data on a visible light image taken by a visible light camera;
obtaining thermal image data that is data on an image taken by an infrared camera having an imaging range corresponding to at least a part of the visible light image;
identifying a person included in the visible light image data and the thermal image data;
obtaining attribute information on the identified person, from a storage apparatus configured to store the attribute information;
obtaining temperature information about a body surface temperature of the person, based on the thermal image data;
setting a threshold, based on the attribute information; and
determining whether the body surface temperature of the person exceeds the threshold or not and outputting determination result information based on a result of determination.

16. A non-transitory computer-readable medium storing an information control program causing a computer to execute:
a process of obtaining visible light image data that is data on a visible light image taken by a visible light camera;
a process of obtaining thermal image data that is data on an image taken by an infrared camera having an imaging range corresponding to at least a part of the visible light image;
a process of identifying a person included in the visible light image data and the thermal image data;
a process of obtaining attribute information on the identified person, from a storage apparatus configured to store the attribute information;
a process of obtaining temperature information about a body surface temperature of the person, based on the thermal image data;
a process of setting a threshold, based on the attribute information; and
a process of determining whether the body surface temperature of the person exceeds the threshold or not and outputting determination result information based on a result of determination.
